(19) United States
(12) Patent Application Publication
(10) Pub. No.: US 2024/0041819 A1
(43) Pub. Date: Feb. 8, 2024

(54) SYSTEMS AND METHODS FOR ENHANCED FINE PARTICULATE DELIVERY IN FEED FOR ANIMALS

(71) Applicant: SarTec Corporation, Anoka, MN (US)

(72) Inventors: Clayton V. McNeff, Andover, MN (US); Larry C. McNeff, Anoka, MN (US); Peter G. Greuel, Anoka, MN (US); Emily Miller, Mounds View, MN (US)

(21) Appl. No.: 18/227,406

(22) Filed: Jul. 28, 2023

Related U.S. Application Data
(63) Continuation-in-part of application No. 16/268,045, filed on Feb. 5, 2019.
(60) Provisional application No. 62/777,999, filed on Dec. 11, 2018.
(60) Provisional application No. 62/626,300, filed on Feb. 5, 2018.
(60) Provisional application No. 63/393,160, filed on Jul. 28, 2022.

(51) Int. Cl. A61K 31/351; A61K 9/16; A61K 9/00; A23K 40/10; A23K 20/121; A23K 10/30; A23K 20/195; A23K 50/10; A23N 17/00

(52) U.S. Cl. CPC A61K 31/351; A61K 9/16; A61K 9/0056; A23K 40/10; A23K 20/121; A23K 10/30; A23K 20/195; A23K 50/10; A23N 17/007

(57) ABSTRACT

Embodiments include animal feed preparation systems and methods. In an embodiment, a method of administering fine particulates with a total mixed ration includes adding total mixed ration components to a vessel. The method further includes adding the fine particulates and an aqueous composition to the vessel. The method further includes mixing the aqueous composition, the fine particulates, and the total mixed ration together to form an agglomerated total mixed ration. The method further includes providing the agglomerated total mixed ration to an animal. Other embodiments are also included herein.

This application is a continuation-in-part application of prior U.S. application Ser. No. 16/268,045, filed Feb. 5, 2019, which claims priority to U.S. Provisional Application Nos. 62/777,999, filed Dec. 11, 2018, and 62/626,300 filed Feb. 5, 2018, the contents of all of which are herein incorporated by reference. This application also claims the benefit of U.S. Provisional Application No. 63/393,160, filed Jul. 28, 2022, the content of which is herein incorporated by reference in its entirety.

### FIELD OF THE TECHNOLOGY

Embodiments herein relate to animal feed preparation systems and methods. More specifically, embodiments herein relate to animal feed preparation systems incorporating the use of an aqueous composition to enhance delivery of fine particulates and related methods.

### BACKGROUND

Animal feed is typically made from large amounts of grain including corn, sorghum, soybeans, wheat, oats, and barley. To ensure animals are receiving complete nutrition including necessary nutrients, various additives (vitamins, minerals, active agents, etc.) can be added to the feed. In many cases these additives are small, fine particulates. The entirety of what an animal is fed can be mixed to form what is known as a total mixed ration (TMR).

### SUMMARY

Embodiments herein relate to animal feed preparation systems incorporating the use of an aqueous composition to enhance delivery of fine particulates and related methods. In a first aspect, a method of administering fine particulates with a total mixed ration can be included, the method including adding total mixed ration components to a vessel, adding the fine particulates to the vessel, adding an aqueous composition to the vessel, mixing the aqueous composition, the fine particulates, and the total mixed ration together to form an agglomerated total mixed ration, and providing the agglomerated total mixed ration to an animal.

In a second aspect, in addition to one or more of the preceding or following aspects, or in the alternative to some aspects, the aqueous composition can further include saponins.

In a third aspect, in addition to one or more of the preceding or following aspects, or in the alternative to some aspects, the aqueous composition can further include sarsasaponins.

In a fourth aspect, in addition to one or more of the preceding or following aspects, or in the alternative to some aspects, the aqueous composition can further include a yucca extract.

In a fifth aspect, in addition to one or more of the preceding or following aspects, or in the alternative to some aspects, mixing the aqueous composition, the fine particulates, and the total mixed ration together to form an agglomerated total mixed ration in the presence of the aqueous composition agglomerates particles to increase the average particle size within the agglomerated total mixed ration.

In a sixth aspect, in addition to one or more of the preceding or following aspects, or in the alternative to some aspects, mixing the aqueous composition, the fine particulates, and the total mixed ration together to form an agglomerated total mixed ration in the presence of the aqueous composition increases the average particle size within the total mixed ration by at least about 10 percent.

In a seventh aspect, in addition to one or more of the preceding or following aspects, or in the alternative to some aspects, mixing the aqueous composition, the fine particulates, and the total mixed ration together to form an agglomerated total mixed ration in the presence of the aqueous composition increases the average particle size within the total mixed ration by at least about 50 percent.

In an eighth aspect, in addition to one or more of the preceding or following aspects, or in the alternative to some aspects, the fine particulates have an average particle size of less than 850, 500, 212, or 150 microns before being added to the vessel.

In a ninth aspect, in addition to one or more of the preceding or following aspects, or in the alternative to some aspects, the fine particulates can include at least one selected from the group consisting of a mineral, a vitamin, an ionophore, a nutritional additive, and a pharmacological agent.

In a tenth aspect, in addition to one or more of the preceding or following aspects, or in the alternative to some aspects, the fine particulates can include monensin.

In an eleventh aspect, in addition to one or more of the preceding or following aspects, or in the alternative to some aspects, the fine particulates can include hydrophobic particles.

In a twelfth aspect, in addition to one or more of the preceding or following aspects, or in the alternative to some aspects, adding the aqueous composition to the vessel can be performed in an amount equal to at least 2.5% by weight of the total mixed ration components.

In a thirteenth aspect, in addition to one or more of the preceding or following aspects, or in the alternative to some aspects, adding the aqueous composition to the vessel can be performed in an amount equal to at least 5% by weight of the total mixed ration components.

In a fourteenth aspect, in addition to one or more of the preceding or following aspects, or in the alternative to some aspects, adding the aqueous composition to the vessel can be performed in an amount equal to at least 7.5% by weight of the total mixed ration components.

In a fifteenth aspect, in addition to one or more of the preceding or following aspects, or in the alternative to some aspects, adding the aqueous composition to the vessel can be performed in an amount equal to at least 10% by weight of the total mixed ration components.

In a sixteenth aspect, in addition to one or more of the preceding or following aspects, or in the alternative to some aspects, providing the agglomerated total mixed ration to the animal includes dispensing the agglomerated total mixed ration into a feed bunk.

In a seventeenth aspect, in addition to one or more of the preceding or following aspects, or in the alternative to some aspects, providing the agglomerated total mixed ration to the animal can be performed no more than 360 minutes after the mixing the aqueous composition, the fine particulates, and the total mixed ration together to form an agglomerated total mixed ration.

In an eighteenth aspect, in addition to one or more of the preceding or following aspects, or in the alternative to some aspects, providing the agglomerated total mixed ration to the animal can be performed no more than 60 minutes after the mixing the aqueous composition, the fine particulates, and the total mixed ration together to form an agglomerated total mixed ration.

In a nineteenth aspect, in addition to one or more of the preceding or following aspects, or in the alternative to some aspects, providing the agglomerated total mixed ration to the animal can be performed no more than 15 minutes after the mixing the aqueous composition, the fine particulates, and the total mixed ration together to form an agglomerated total mixed ration.

In a twentieth aspect, in addition to one or more of the preceding or following aspects, or in the alternative to some aspects, mixing the aqueous composition, the fine particulates, and the total mixed ration together to form an agglomerated total mixed ration can be performed on a mobile platform.

In a twenty-first aspect, in addition to one or more of the preceding or following aspects, or in the alternative to some aspects, the method can further include measuring the moisture content of the agglomerated total mixed ration.

In a twenty-second aspect, in addition to one or more of the preceding or following aspects, or in the alternative to some aspects, the animal can include a ruminant.

In a twenty-third aspect, in addition to one or more of the preceding or following aspects, or in the alternative to some aspects, the animal can include Bos taurus.

In a twenty-fourth aspect, in addition to one or more of the preceding or following aspects, or in the alternative to some aspects, the total mixed ration components can include from 1 to 15 wt. percent (dry matter basis) fat content.

In a twenty-fifth aspect, in addition to one or more of the preceding or following aspects, or in the alternative to some aspects, the total mixed ration components can include from 2 to 10 wt. percent (dry matter basis) fat content.

In a twenty-sixth aspect, in addition to one or more of the preceding or following aspects, or in the alternative to some aspects, the total mixed ration components can include from 3 to 6 wt. percent (dry matter basis) fat content.

In a twenty-seventh aspect, a method of increasing consistency of fine particulate dosing to animals exhibiting feed sorting behavior can be included. The method can include adding total mixed ration components to a vessel, adding fine particulates to the vessel, adding an aqueous composition to the vessel, mixing the aqueous composition, the fine particulates, and the total mixed ration together to form a mixed total mixed ration, and providing the mixed total mixed ration to an animal.

In a twenty-eighth aspect, in addition to one or more of the preceding or following aspects, or in the alternative to some aspects, the aqueous composition can include a surfactant.

In a twenty-ninth aspect, in addition to one or more of the preceding or following aspects, or in the alternative to some aspects, the aqueous composition can include saponins.

In a thirtieth aspect, in addition to one or more of the preceding or following aspects, or in the alternative to some aspects, the aqueous composition can include sarsasaponins.

In a thirty-first aspect, in addition to one or more of the preceding or following aspects, or in the alternative to some aspects, the aqueous composition can include a yucca extract.

In a thirty-second aspect, in addition to one or more of the preceding or following aspects, or in the alternative to some aspects, mixing the aqueous composition, the fine particulates, and the total mixed ration together to form a mixed total mixed ration in the presence of the aqueous composition agglomerates particles to increase the average particle size within the total mixed ration.

In a thirty-third aspect, in addition to one or more of the preceding or following aspects, or in the alternative to some aspects, mixing the aqueous composition, the fine particulates, and the total mixed ration together to form a mixed total mixed ration in the presence of the aqueous composition increases the average particle size within the total mixed ration by at least about 10 percent.

In a thirty-fourth aspect, in addition to one or more of the preceding or following aspects, or in the alternative to some aspects, mixing the aqueous composition, the fine particulates, and the total mixed ration together to form a mixed total mixed ration in the presence of the aqueous composition increases the average particle size within the total mixed ration by at least about 50 percent.

In a thirty-fifth aspect, in addition to one or more of the preceding or following aspects, or in the alternative to some aspects, the fine particulates have an average particle size of less than 850, 500, 212, or 150 microns before being added to the vessel.

In a thirty-sixth aspect, in addition to one or more of the preceding or following aspects, or in the alternative to some aspects, the fine particulates can include at least one selected from the group consisting of a mineral, a vitamin, an ionophore, a nutritional additive, and a pharmacological agent.

In a thirty-seventh aspect, in addition to one or more of the preceding or following aspects, or in the alternative to some aspects, the fine particulates can include monensin.

In a thirty-eighth aspect, in addition to one or more of the preceding or following aspects, or in the alternative to some aspects, the fine particulates can include hydrophobic particles.

In a thirty-ninth aspect, in addition to one or more of the preceding or following aspects, or in the alternative to some aspects, adding the aqueous composition to the vessel can be performed in an amount equal to at least 2.5% by weight of the total mixed ration components.

In a fortieth aspect, in addition to one or more of the preceding or following aspects, or in the alternative to some aspects, adding the aqueous composition to the vessel can be performed in an amount equal to at least 5% by weight of the total mixed ration components.

In a forty-first aspect, in addition to one or more of the preceding or following aspects, or in the alternative to some aspects, adding the aqueous composition to the vessel can be performed in an amount equal to at least 7.5% by weight of the total mixed ration components.

In a forty-second aspect, in addition to one or more of the preceding or following aspects, or in the alternative to some aspects, adding the aqueous composition to the vessel can be performed in an amount equal to at least 10% by weight of the total mixed ration components.

In a forty-third aspect, in addition to one or more of the preceding or following aspects, or in the alternative to some aspects, providing the mixed total mixed ration to the animal includes dispensing the mixed total mixed ration into a feed bunk.

In a forty-fourth aspect, in addition to one or more of the preceding or following aspects, or in the alternative to some aspects, providing the mixed total mixed ration to the animal can be performed no more than 360 minutes after the mixing the aqueous composition, the fine particulates, and the total mixed ration together to form a mixed total mixed ration.

In a forty-fifth aspect, in addition to one or more of the preceding or following aspects, or in the alternative to some aspects, providing the mixed total mixed ration to the animal can be performed no more than 60 minutes after the mixing the aqueous composition, the fine particulates, and the total mixed ration together to form a mixed total mixed ration.

In a forty-sixth aspect, in addition to one or more of the preceding or following aspects, or in the alternative to some aspects, providing the mixed total mixed ration to the animal can be performed no more than 15 minutes after the mixing the aqueous composition, the fine particulates, and the total mixed ration together to form a mixed total mixed ration.

In a forty-seventh aspect, in addition to one or more of the preceding or following aspects, or in the alternative to some aspects, mixing the aqueous composition, the fine particulates, and the total mixed ration together to form a mixed total mixed ration can be performed on a mobile platform.

In a forty-eighth aspect, in addition to one or more of the preceding or following aspects, or in the alternative to some aspects, the method can further include measuring the moisture content of the mixed total mixed ration.

In a forty-ninth aspect, in addition to one or more of the preceding or following aspects, or in the alternative to some aspects, the animal can include a ruminant.

In a fiftieth aspect, in addition to one or more of the preceding or following aspects, or in the alternative to some aspects, the animal can include Bos taurus.

In a fifty-first aspect, in addition to one or more of the preceding or following aspects, or in the alternative to some aspects, the method can further include selecting an animal exhibiting feed sorting behavior.

In a fifty-second aspect, in addition to one or more of the preceding or following aspects, or in the alternative to some aspects, the total mixed ration components can include from 1 to 15 wt. percent (dry matter basis) fat content.

In a fifty-third aspect, in addition to one or more of the preceding or following aspects, or in the alternative to some aspects, the total mixed ration components can include from 2 to 10 wt. percent (dry matter basis) fat content.

In a fifty-fourth aspect, in addition to one or more of the preceding or following aspects, or in the alternative to some aspects, the total mixed ration components can include from 3 to 6 wt. percent (dry matter basis) fat content.

In a fifty-fifth aspect, an animal feed preparation system can be included having a control circuit and a vessel, wherein the vessel can be configured to receive total mixed ration components. The system can also include a moisture sensor, wherein the moisture sensor can be configured to measure moisture content within the vessel. The system can also include an aqueous composition supply, wherein the aqueous composition supply can be configured to be controlled by the control circuit and to inject the aqueous composition into the vessel. The system can also include a fine particulate dosing device, wherein the fine particulate dosing device can be configured to supply doses of fine particulate material into the vessel. The animal feed preparation system can be configured to mix the aqueous composition, the fine particulates, and the total mixed ration components together to form an agglomerated total mixed ration with an average particle size larger than the particle size of the starting materials.

In a fifty-sixth aspect, in addition to one or more of the preceding or following aspects, or in the alternative to some aspects, the aqueous composition can include a surfactant.

In a fifty-seventh aspect, in addition to one or more of the preceding or following aspects, or in the alternative to some aspects, the aqueous composition can include saponins.

In a fifty-eighth aspect, in addition to one or more of the preceding or following aspects, or in the alternative to some aspects, the aqueous composition can include sarsasaponins.

In a fifty-ninth aspect, in addition to one or more of the preceding or following aspects, or in the alternative to some aspects, the aqueous composition can include a yucca extract.

In a sixtieth aspect, in addition to one or more of the preceding or following aspects, or in the alternative to some aspects, the agglomerated total mixed ration can have an average particle size at least about 10 percent than the particle size of the starting materials.

In a sixty-first aspect, in addition to one or more of the preceding or following aspects, or in the alternative to some aspects, the agglomerated total mixed ration can have an average particle size at least about 50 percent than the particle size of the starting materials.

In a sixty-second aspect, in addition to one or more of the preceding or following aspects, or in the alternative to some aspects, the fine particulates have an average particle size of less than 850, 500, 212, or 150 microns before being added to the vessel.

In a sixty-third aspect, in addition to one or more of the preceding or following aspects, or in the alternative to some aspects, the fine particulates can include at least one selected from the group consisting of a mineral, a vitamin, an ionophore, a nutritional additive, and a pharmacological agent.

In a sixty-fourth aspect, in addition to one or more of the preceding or following aspects, or in the alternative to some aspects, the fine particulates can include monensin.

In a sixty-fifth aspect, in addition to one or more of the preceding or following aspects, or in the alternative to some aspects, the fine particulates can include hydrophobic particles.

In a sixty-sixth aspect, in addition to one or more of the preceding or following aspects, or in the alternative to some aspects, the animal feed preparation system can be configured to add the aqueous composition in an amount equal to at least 2.5% by weight of the total mixed ration components.

In a sixty-seventh aspect, in addition to one or more of the preceding or following aspects, or in the alternative to some aspects, the animal feed preparation system can be configured to add the aqueous composition in an amount equal to at least 5% by weight of the total mixed ration components.

In a sixty-eighth aspect, in addition to one or more of the preceding or following aspects, or in the alternative to some aspects, the animal feed preparation system can be configured to add the aqueous composition in an amount equal to at least 7.5% by weight of the total mixed ration components.

In a sixty-ninth aspect, in addition to one or more of the preceding or following aspects, or in the alternative to some aspects, the animal feed preparation system can be configured to add the aqueous composition in an amount equal to at least 10% by weight of the total mixed ration components.

In a seventieth aspect, in addition to one or more of the preceding or following aspects, or in the alternative to some aspects, the animal feed preparation system can be mounted on a mobile platform.

In a seventy-first aspect, in addition to one or more of the preceding or following aspects, or in the alternative to some aspects, the total mixed ration components can include from 1 to 15 wt. percent (dry matter basis) fat content.

In a seventy-second aspect, in addition to one or more of the preceding or following aspects, or in the alternative to some aspects, the total mixed ration components can include from 2 to 10 wt. percent (dry matter basis) fat content.

In a seventy-third aspect, in addition to one or more of the preceding or following aspects, or in the alternative to some aspects, the total mixed ration components can include from 3 to 6 wt. percent (dry matter basis) fat content.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope of the present application is defined by the appended claims and their legal equivalents.

### BRIEF DESCRIPTION OF THE FIGURES

The technology may be more completely understood in connection with the following drawings, in which:

FIG. 1 is a schematic diagram of particulates in a vessel, according to an embodiment.

FIG. 2 is a schematic diagram of particulates in a vessel, according to an embodiment.

FIG. 3 is a flowchart showing a method of preparing animal feed within a vessel in accordance with embodiments herein.

FIG. 4 is a flowchart showing a method of preparing animal feed within a vessel in accordance with embodiments herein.

FIG. 5 is a schematic view of a moisture addition system, according to an embodiment.

FIG. 6 is a schematic view of a moisture addition system, according to an embodiment.

FIG. 7 is a schematic view of a mobile moisture addition system, according to an embodiment.

FIG. 8 is a schematic diagram of a moisture addition system in accordance with various embodiments herein.

FIG. 9 is a schematic diagram of a moisture addition system in accordance with various embodiments herein.

FIG. 10 is graph showing a comparison of mass percent of particulate size in total mixed rations (TMR) based on the percentage of moisture added.

FIG. 11 is graph showing a comparison of mass percent of particulate size in total mixed rations (TMR) based on the percentage of moisture added.

FIG. 12 is a graph showing the impact of equilibration time on fines reduction.

While the technology is susceptible to various modifications and alternative forms, specifics thereof have been shown by way of example and drawings, and will be described in detail. It should be understood, however, that the application is not limited to the particular embodiments described. On the contrary, the application is to cover modifications, equivalents, and alternatives falling within the spirit and scope of the technology.

### DETAILED DESCRIPTION

In some cases, animals may be feed with a total mixed ration (TMR). A total mixed ration is the result of weighing and blending all feed stuffs into a complete ration which provides adequate nourishment for a given animal and may be their sole source of feed. However, as described above, sometimes additives are provided to ensure proper nutrition that may take the form of fine particulates. Unfortunately, fine particulates may separate out from other components of the TMR and fall to the bottom of the feed bunk or other feeding station. This can lead to costly ingredients not being consumed by the animals. This is especially problematic for animals that exhibit food sorting behavior. Animals exhibiting food sorting behavior will often sort through the feed and pick out the larger, coarser grain particulates while leaving behind the smaller, finer particulates. This can result in some animals receiving too little of the additives, with other animals, such as those coming later, receiving too much of the finer particulates.

In addition, the TMR is typically mechanically mixed. However, the action of mixing can generate more fine particulates as materials interface with and may be mechanically broken up by a stirring mechanism. Further, as cattle consume the total mixed ration the differently sized particles can become separated from each other for both chemical and mechanical reasons and the result is that fine particles can separate from the larger particle in the ration and create localized difference in the nutritional content of ration. The smaller particles or “fines” usually end up not being consumed by the cattle initially and can end up at the bottom of a feed bunk where they are eventually consumed. Overall, the result for particle separation in a total mixed ration can result in a distortion of the nutritional value of the ration that cattle consume at different times as the total mixture is consumed. The overall effect of these phenomena is to create a distortion targeted ration that is represented by the formulation of the total mixed ration for different cattle in a group.

However, methods herein and compositions used in the same (such as yucca-based feed additives that contain naturally occurring surfactants called saponins) can significantly reduce the separation of fine particles from larger particles and the resulting nutritional distortion that can naturally occur with a TMR. Data herein shows that such methods including mixing such compositions into the ration halts the falling apart of small particles (including, but not limited to, added pharmaceuticals) from the larger particles in the ration so that a better and more consistent average composition is consumed by the cattle over the feeding period.

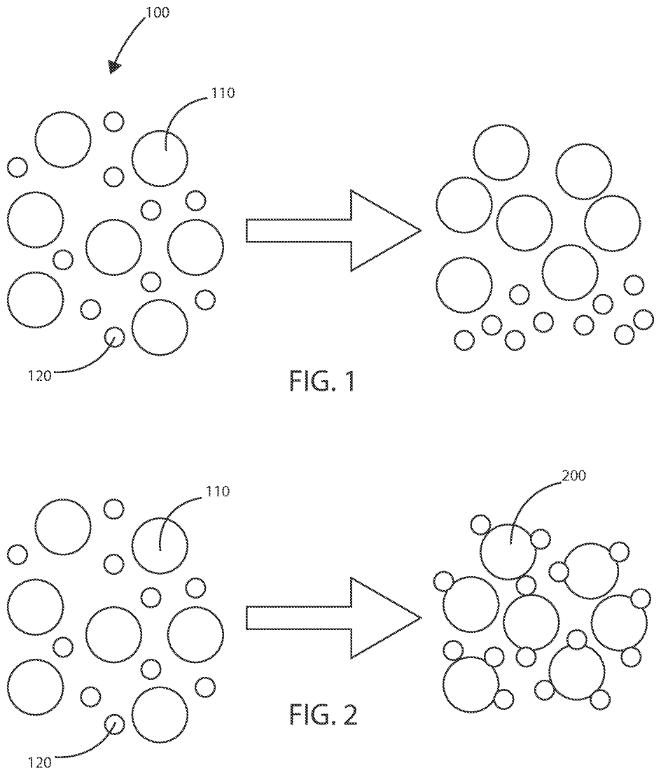

FIG. 1 shows a schematic diagram 100 of particulates within a vessel. FIG. 1 illustrates the problem of mixing smaller, finer particulates in with larger, coarser particulates. Specifically, after larger particulates 110 and finer particulates 120 are mixed together the finer particulates 120 can settle out on the bottom of a vessel in which they are mixed and/or in the bottom of a feed bunk or other feeding station. This can lead to some animals eating more of the larger particulates 110 when they feed, while other animals may eat more of the finer particulates 120. This can also lead to the finer particulates 120 being left at the bottom of the feed container without being consumed.

Additionally, animals exhibiting food sorting behavior can often exacerbate the problem of finer particulates 120 settling on the bottom of the feed container. Specifically, animals exhibiting feed sorting behavior will often sort through the feed provided in search of the larger particulates 110 they deem more desirable to eat. This can cause the finer particulates 120 to be pushed aside and settle at the bottom of the feed bunk or other feeding station.

However, embodiments herein can include systems and methods for preventing fine particulates from settling at the bottom of a feed container or otherwise being sorted out by animals exhibiting feed sorting behavior. In various embodiments, total mixed ration, fine particulates, and an aqueous composition can be added to a vessel and mixed together in order to create an agglomerated total mixed ration. In the agglomerated total mixed ration, the previous fine particulates can adhere to larger particulates increasing the average size of particulates in the total mixed ration. The previous fine particulates can remain adhered to larger particulates after the total mixed ration is deposited in the feed bunk such that such that animals eating larger particulates will also receive the content of the fine particulates.

FIG. 2 shows a schematic diagram of particulates within a vessel, according to various embodiments herein. As shown, larger particulates 110 and finer particulates 120 can be mixed together and an aqueous composition can be added to the mixture in order to create larger, agglomerated particulates 200. The agglomerated particulates 200 include the finer particulates 120 bound to the larger particulates 110. This prevents animals from sorting and separating the larger particulates 110 out from the finer particulates 120. Additionally, this approach can serve to prevent the creation of additional finer particulates 120 during mixing processes as the aqueous composition can effectively serve as a lubricating agent. Further, by adhering fine particulates to larger particulates, the finer particulates 120 are prevented from settling on the bottom of the feed bunk thereby allowing each animal to receive a more consistent amount of the finer particulates 120.

### Methods

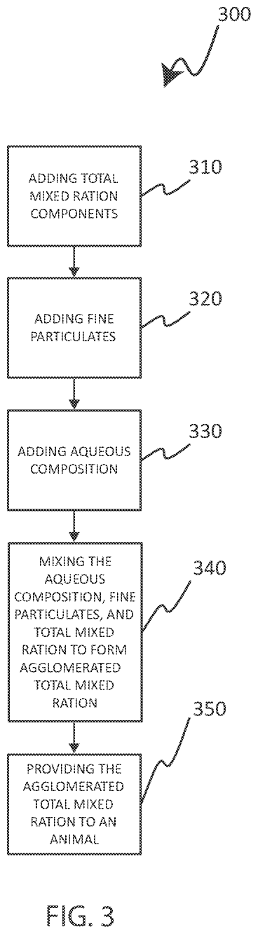

Various methods are included herein. In some embodiments, a method of administering fine particulates with a total mixed ration is included. FIG. 3 shows a flow chart showing a method 300 of preparing animal feed within a vessel according to an embodiment.

In some embodiments, the method 300 can include adding total mixed ration components to a vessel 310. In some embodiments, the method 300 can include adding fine particulates to the vessel 320. The fine particulates can be added in a dry form, as a slurry in water, or even in the form of a liquid supplement. In some embodiments, the fine particulates can have an average particle size (by weight) of equal to or less than about 850, 500, 212, or 150 microns, or can fall within a range between any of the foregoing. In some cases, operation 320 can occur simultaneously with operation 310. In some cases, operation 320 can occur simultaneously with operation 330 discussed below. In some cases, operations 310, 320, and 330 and all occur simultaneously, but in other cases the operations can be performed at separate times. As such, the order of addition of the components can be changed. In some embodiments, the total mixed ration components, the fine particulates, and the aqueous composition can all be added at approximately the same time. In some embodiments, at least a portion of the fine particulates can be added at the same time as at least a portion of the aqueous composition is being added. In other embodiments, the fine particulates are added to the vessel prior to any aqueous composition being added to the vessel.

The method 300 can include adding an aqueous composition to the vessel 330. In some embodiments, the amount of aqueous composition is an amount equal to at least 1%, 2%, 3%, 4%, 5%, 6%, 7%, 8%, 9%, 10%, 11%, 12%, 13%, 14%, 15%, 16%, 17%, 18%, 19%, 20% by weight of the total mixed ration components, or can fall within a range between any of the foregoing. In some embodiments, the amount of aqueous composition is an amount equal to at least 2.5% by weight of the total mixed ration components. In some embodiments, the amount of aqueous composition is an amount equal to at least 5% by weight of the total mixed ration components. In some embodiments, the amount of aqueous composition is an amount equal to at least 7.5% by weight of the total mixed ration components. In some embodiments, the amount of aqueous composition is an amount equal to at least 10% by weight of the total mixed ration components.

In various embodiments, an aqueous composition supply controls the rate and/or amount of aqueous composition being added to the vessel. The aqueous composition can contain water and one or more additives such as those described elsewhere herein. In some embodiments, the aqueous composition can contain an amount of the one or more additives sufficient for the fine particulates to agglomerate to the total mixed ration components in the vessel. In some embodiments, the total amount of aqueous composition to be added to the vessel can be calculated prior adding any of the aqueous composition to the vessel, such as based on the amount of the total mixed ration and fine particulates that the aqueous composition will be mixed with, the initial moisture content of the total mixed ration, and a desired moisture content of the total mixed ration.

The method 300 can include mixing the aqueous composition, the fine particulates, and the total mixed ration together to form an agglomerated total mixed ration 340. In some embodiments, mixing the aqueous composition, the fine particulates, and the total mixed rations together is performed on a mobile platform, described in greater detail below.

In various embodiments, mixing the total mixed ration and fine particulates together with the aqueous composition agglomerates particles to increase the average particle size within the total mixed ration. In some embodiments, the average particle size within the total mixed ration increases by at least 5, 10, 15, 20, 25, 30, 35, 40, 45, 50, 55, 60, 65, 70, 75, 100, 150, 200, 300, or 500 percent or more, or can fall within a range between any of the foregoing. In some embodiments, the average particle size within the total mixed ration increases by at least about 10 percent. In some embodiments, the average particle size within the total mixed ration increases by at least about 50 percent.

The method 300 can include providing the agglomerated total mixed ration to an animal 350. In some embodiments, the animal can include ruminants such as Bos taurus, sheep, goats, bison, buffalo, deer, and antelopes. In some embodiments, the agglomerated total mixed ration can be provided to an animal no more than 10 minutes, 20 minutes, 30 minutes, 40 minutes, 50 minutes, 60 minutes, 70 minutes, 80 minutes, 90 minutes, 120 minutes, 240 minutes, 360 minutes, or 600 minutes after mixing the aqueous composition, the fine particulates, and the total mixed ration together, or can fall within a range between any of the foregoing. In some embodiments, the agglomerated total mixed ration can be provided to an animal no more than 60 minutes after mixing the aqueous composition, the fine particulates, and the total mixed ration together. In some embodiments, the agglomerated total mixed ration can be provided to an animal no more than 15 minutes after mixing the aqueous composition, the fine particulates, and the total mixed ration together.

In some embodiments, the method 300 can include measuring the moisture content of the mixed total mixed ration, described in greater detail below.

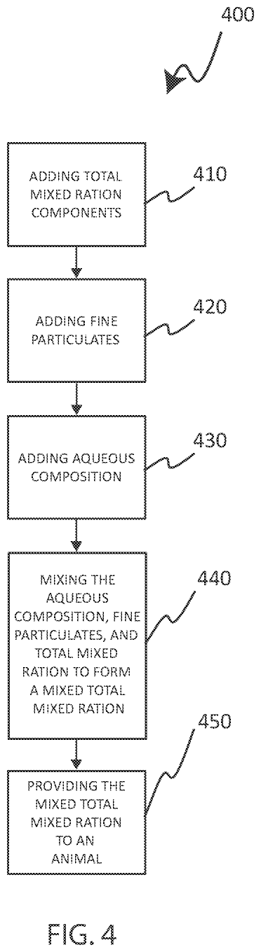

In some embodiments, a method of increasing consistency of fine particulate dosing to animals exhibiting feed sorting behavior is included. FIG. 4 shows a flow chart showing a method 400 of preparing animal feed within a vessel according to an embodiment.

In some embodiments, the method 400 can include adding total mixed ration components to a vessel 410. In some embodiments, the method 400 can include adding fine particulates to the vessel 420. The fine particulates can be added in a dry form, as a slurry in water, or even in the form of a liquid supplement. In some embodiments, the fine particulates can have an average particle size of about less than 850, 500, 212, or 150 microns, or can fall within a range between any of the foregoing. In some embodiments, at least a portion of the fine particulates can be added at the same time as at least a portion of the aqueous composition is being added. In other embodiments, the fine particulates are added to the vessel prior to any aqueous composition being added to the vessel. However, it will be appreciated that the order of addition of components can be varied. As such, in some cases, operation 420 can occur simultaneously with operation 410. In some cases, operation 420 can occur simultaneously with operation 430 discussed below. In some cases, operations 410, 420, and 430 and all occur simultaneously, but in other cases the operations can be performed at separate times.

The method 400 can include adding an aqueous composition to the vessel 430 in a manner and in amounts similar to that as described with respect to FIG. 3.

In various embodiments, an aqueous composition supply controls the rate and/or amount of aqueous composition being added to the vessel. The aqueous composition can contain water and one or more additives such as those described elsewhere herein. In some embodiments, the aqueous composition can contain an amount of the one or more additives sufficient for the fine particulates to agglomerate to the total mixed ration components in the vessel. In some embodiments, the total amount of aqueous composition to be added to the vessel can be calculated prior adding any of the aqueous composition to the vessel, such as based on the amount of the total mixed ration and fine particulates that the aqueous composition will be mixed with, the initial moisture content of the total mixed ration, and a desired moisture content of the total mixed ration.

The method 400 can include mixing the aqueous composition, the fine particulates, and the total mixed ration together to form a mixed total mixed ration 440 in a manner similar to that as described with respect to FIG. 3. The method 400 can also include providing the mixed total mixed ration to an animal 450 in a manner similar to that as described with respect to FIG. 3. In some embodiments, the method 400 can include measuring the moisture content of the mixed total mixed ration, described in greater detail below.

Systems and methods for adding moisture, and in some cases additives with the moisture, to feedstuff materials such as grains, other feed stuffs, and total mixed rations are described herein. Materials to which moisture can be added can include grains and specifically feed grains. Materials to which moisture can be added can include, but are not limited to, corn, wheat, millet, milo (grain sorghum), oats, barley, rye, spelt, triticale, amaranth, buckwheat, rice, soybeans, sunflower seeds, other oil seeds. Materials to which moisture can be added can also include forage materials including, but not limited to hay, sillage, stover, and the like. Other materials can include other types of plant matter besides those described above, other natural particulates, synthetic particulates, mineral based particulates, other byproduct animal feed materials such as molasses, beet pulp, distillers grains, brewers grains, corn gluten feed, and the like. In various embodiments, materials here can specifically include components of a total mixed ration. Details regarding total mixed rations are described in greater detail below.

### Moisture Addition Systems

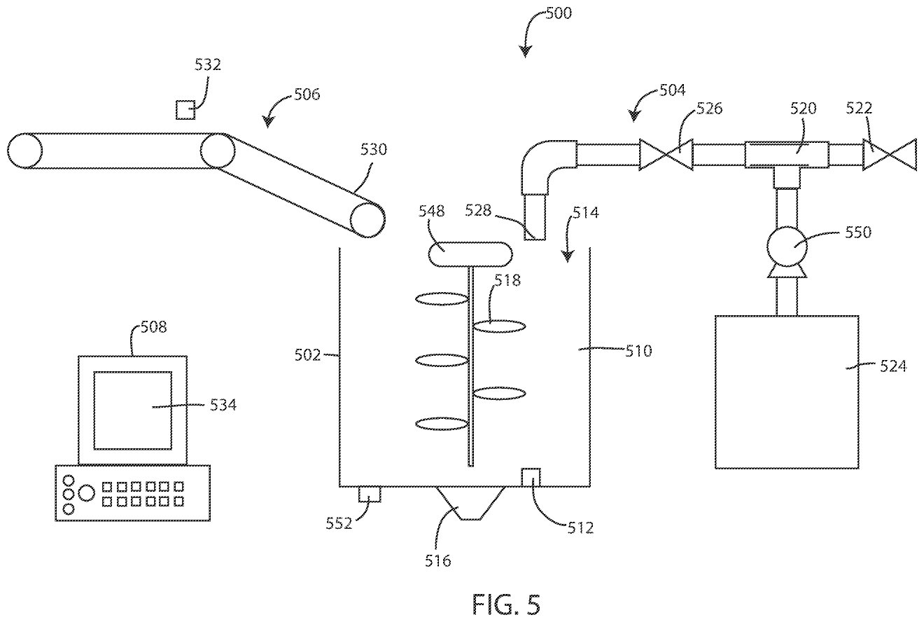

In reference now to the figures, FIG. 5 shows a schematic view of an aqueous composition addition system 500. In an embodiment, the aqueous composition addition system 500 can include a vessel 502, an aqueous composition supply 504, a feedstuff supply system 506, and a control circuit 508.

The vessel 502 can define an interior volume 510. The interior volume 510 can be configured to hold or store the feed stuffs being conditioned. The aqueous composition supply 504 can be configured to deliver aqueous composition into the interior volume 510, such as to be mixed with the feed stuffs. In some embodiments, the aqueous composition injects the aqueous composition into the vessel 502. The feedstuff supply system 506 can be configured to deliver feed stuffs into the interior volume 510, such as to be mixed with the aqueous composition. The control circuit 508 can be configured to monitor the moisture content of the material within the interior volume 510, as well as to control the addition of feedstuffs and/or aqueous composition into the vessel 502.

### Vessel

In some embodiments, various components can be disposed within the interior volume 510 of the vessel 502. In some embodiments, a first moisture sensor 512 can be disposed within the interior volume 510. The first moisture sensor 512 can be configured to measure the moisture content of the material within the vessel 502. The first moisture sensor 512 can measure the moisture content of the material within the vessel while aqueous composition and/or feed stuffs are being added to the interior volume 510.

In various embodiments, the first moisture sensor 512 can include a microwave moisture sensor. By way of example, a material to be measured can be passed across the microwave moisture sensor which radiates an extremely low powered electromagnetic field. Due to the dipolar effect of a water molecule, the resonant frequency of a microwave resonator changes with variations in moisture content. It is these variations that are detected by the sensor electronics. They are then measured in terms of ‘unscaled units’ which are scaled by a process of calibration to provide a precise readout of the moisture present. The resulting signal can be sent via an analogue (0-20 mA [0-10 v] or 4-20 mA) or RS485 digital communications link to other system components, such as a control circuit. One example of a microwave moisture sensor is a digital microwave moisture sensor produced by Hydronic® based in Guildford, United Kingdom. The first moisture sensor 512 can be configured to send a signal which represents the moisture content of the material in the vessel 502 to the control circuit 508. In some embodiments, the moisture of the material in the interior volume at the start of the process is about 4, 5, 6, 7, 8, 9, 10, 11, 12, 13, 14, 15, 16, 17, 18, 19, 20, 21 or 22 percent by weight, or can fall within a range between any of the foregoing.

The vessel 502 can include an opening 514. Material, such as unconditioned feed stuffs and aqueous composition, can be added to the interior volume 510 through the opening 514. The vessel 502 can include an outlet 516. The outlet 516 can be configured to allow material, such as conditioned feedstuffs, to exit the vessel 502. In some embodiments, the outlet 516 can include a valve which can control the rate at which material exits the vessel 502. In various embodiments, the outlet 516 can be located at the bottom of the vessel 502, such that gravity can aid in moving material out of the interior volume 510 through the outlet 516.

In some embodiments, a mixing apparatus 518 can be disposed within the interior volume 510. The mixing apparatus 518 can be configured to mix material within the interior volume to ensure material is homogeneous, such as be ensuring the feed stuffs are sufficiently mixed with the aqueous composition. In some embodiments, the mixing apparatus 518 can include a mechanical mixing element, such as a rotating shaft and blades or paddles. In some embodiments, a motor 548 to generate mechanical force to turn the rotating shaft and/or blades or paddles. In some embodiments, the mixing apparatus 518 can include a recirculation pump.

In some embodiments, other sensors can be included beyond moisture sensors. For example, a weight sensor 552 such as a load cell can be included and can be used by the system, for example, to verify the addition of materials to the vessel 502. In some embodiments, the weight sensor 552 can be used in combination with the first moisture sensor 512. For example, the feedstuffs can be added to the vessel and the total amount weighed, then the moisture sensor (before, during or after mixing) can take a measurement of the moisture content, which can then be compared to a target moisture content. Then a precise amount of aqueous composition to be added can be calculated in order to raise the moisture content to the target moisture content.

### Aqueous Composition Supply

The aqueous composition supply 504 can be configured to add into the interior volume 510 of the vessel 502. The aqueous composition can be mixed with the feed stuffs to increase the moisture content of the feed stuffs. In some embodiments, the aqueous composition can include water.

In some embodiments, the aqueous composition can include water and one or more additives. The additive can have various properties including acting as one or more of a binding agent, a fortifying agent, a surfactant, a processing aid, a flavoring, a coloring material, a preservative, of the like. In some embodiments, an anti-protozoal additive can be included. In some embodiments, an additive that reduces methane production can be added.

In some embodiments, a binding composition can be added as an additive. In some embodiments, the binding additive acts to bind the fine particulates to the larger, coarser particulates in the total mixed ration. For example, the binding additive can include molasses, malt syrup, corn syrup, high fructose corn syrup, hydrogenated corn syrup, hydrogenated menhaden oil, and mandarin oil.

In various embodiments, a saponin containing composition can be added as an additive. In some embodiments, the aqueous composition can include sarsasaponins. By way of example, saponins useful in the present invention may also be extracted in sufficient concentrations from plants of the family: Amaryllidaccae, genus: Agave, which grows extensively in the southwestern United States and in Mexico. Saponins useful in the present invention may also be extracted in sufficient concentrations from plants of the family: Lillaecae, genus: Yucca, as well as from Quillaja saponaria bark. Saponins may be extracted from plant materials in accordance with techniques well-known by those of skill in the art.

The Yucca plant is a wide-ranging genus, which is part of the Century plant family, Aguavacea. Taxonomically there are 30 species within the Yucca genus, Schidigera being one. The typical saponin content that naturally occurs in yucca plants is from 0.1-2% saponins by weight. Yucca extracts can be derived by extracting yucca powder with an aqueous solution that may or may not contain some fraction of organic solvent such as methanol, ethanol, propanol, butanol, or the like. Commercially available Yucca extracts can have a total solids content usually in the range from 5-50%.

The saponin content of a typical 50 brix (50% solids by weight) yucca extract is usually in the range of about 1-2% saponins by weight as measured by HPLC analysis. In an embodiment of the invention, the saponin containing composition comprises between about 10-50% (total solids content) yucca extract that contains 1-2% saponins by weight as measured by HPLC analysis. Another method of measuring total saponin content is the extraction of all soluble components into a butanol extract followed by gravimetric analysis of the compounds dissolved in the butanol fraction. Measuring saponin content by the butanol extract method typically results in higher numbers than the more advanced HPLC method. Accordingly, the typical 50 brix (50% solids by weight) yucca extract is usually in the range of about 5-20.0% saponins content by weight as measured by the butanol extract method.

In an embodiment, the saponin containing composition used in accordance with the invention comprises at least 0.1% by weight saponins as measured by HPLC. In an embodiment, the saponin containing composition used in accordance with the invention comprises at least 0.5% by weight saponins as measured by HPLC. In a particular embodiment, the saponin containing composition used in accordance with the invention comprises at least 1.0% by weight saponins as measured by HPLC. It is believed that the effects of the composition are related to the total amount of saponins present. Thus, one of skill in the art will appreciate that if a certain amount of saponins is desired it can be achieved either through varying the volume of a certain concentration composition administered, varying the concentration of a certain volume of a composition, or both.

An exemplary liquid form of a saponin containing composition is sold under the trademark SARIEMP® by SarTec Corporation of Anoka, Minnesota. It is prepared by blending an aqueous extract of the plants of the family: Lillaecae, genus: Yucca, or other appropriate Yucca plants containing 10% solids with antifreeze agents such as calcium chloride, propylene glycol, n-propanol, glycerine, sodium chloride, potassium chloride, and the like, to depress the freezing point to −30° F. Antifreeze can allow the composition to be stored outside in the winter. The final concentration of Yucca soluble solids is 8.25%. Its physical data are:

| Bulk Density | 10.4 lbs. per gallon |
|---|---|
| Color | Dark brown |
| Freezing Point | −30° F. |
| PH | 5.5-6.0 |
| Total solids | 33% |
| Water | 67% |

For some applications herein, this liquid material can be applied at the rate of 1-fluid ounces or about 12 to 185 grams (dry weight) per ton of particulate. In an embodiment, the aqueous composition added to the feedstuffs or other materials can include about 0.1, 0.2, 0.3, 0.4, 0.5, 0.6, 0.7, 0.8, 0.9, 1.0, 1.2, 1.4, 1.6, 1.8, 2.0, 2.4, 2.8, 3.2, 3.6, 4.0, 5, 6, 7, 8, 10, 12, 15, 20 or 25% by weight additive, or the amount can fall within a range between any of the foregoing. In various embodiments, the saponin containing liquid material can be applied at the rate of about 2 ounces per ton of TMR dry matter.

Other exemplary liquid solutions containing saponins are available commercially and sold under the trademarks SAR IEMP®, SARSTART®, SARSTART® PRO, SARSTART® PLUS, and SARSTART® LSC GOLD by SarTec Corporation of Anoka, Minn. These solutions are prepared by blending an aqueous extract of the plants of the family: Lillaecae, genus: Yucca, or other appropriate Yucca plants with antifreeze agents such as calcium chloride, sodium chloride, potassium chloride, propylene glycol, glycerine, and the like, to depress the freezing point to approximately −30° F. These liquid solutions may also comprise a variety of other components. By way of example, SARSTART® PLUS can contain the following ingredients: water, propylene glycol, Yucca schidigera extract, vitamin E (as di-alpha-tocopheryl acetate), vitamin A propionate, vitamin A palmitate, vitamin B1, vitamin B2, vitamin B6, vitamin B12, D-Activated animal sterol (source of Vitamin D3), naturally occurring organisms, dried egg solids, dried casein, and dried whey. The physical and chemical characteristics of SARSTART® PLUS are as follows: Boiling Point: 240° F.; Specific Gravity: 1; Melting Point: −20° F.; Solubility in Water: Miscible; Appearance and Odor: Dark brown liquid with a mild odor and a slightly acid taste.

In various embodiments, the components added to the TMR (such as with the aqueous composition, but also could be added separately) include one or more surfactants. The saponins of saponin containing compositions herein can function as surfactants. Other surfactants that can be used in compositions herein (in addition to or in place of saponin containing compositions) can include, but are not limited to, surfactants that are safe for animal consumption including various ionic surfactants (anionic and cationic), non-ionic surfactants, and the like, including but not limited to various sulfates, sulfonates, quaternary compounds, and the like.

In some embodiments, the aqueous composition can include macroalgae or a component derived therefrom. Macroalgae, commonly referred to as seaweed, includes over 30,000 of species of macroscopic marine algae. Macroalgae herein can include Rhodophyta (red macroalgae), Phaeophyta (brown macroalgae), and Chlorophyta (green macroalgae). In some embodiments the aqueous composition can include a macroalgae selected from the genus of at least one of Asparagopsis, Polysiphonia, Ceramium, Gracilaria, Chondrus, Porphyra, Macrocystis, Fucus, Sargassum, Laminaria, Dictyota, Ascophyllum, Saccharina, Palmaria, Nereocystis, Ulva, Codium, Clad ophora, Caulerpa, Capsosiphon, or Halimeda. In some embodiments, the aqueous composition can include at least 0.001, or 0.01, or 0.1 wt. percent macroalgae or a component derived therefrom.

The aqueous composition supply 504 can be connected to a water supply 520. In some embodiments, the water supply 520 can include connection to a water source, such as shown in FIG. 5. In other embodiments, the water supply 520 can include a water storage tank. The water supply 520 can be connected to a water valve 522 which can control the amount and rate of water being added to the interior volume 510 of the vessel.

The aqueous composition supply 504 can include an additive supply 524. The additive supply 524 can include a tank or storage of additive. The additive supply 524 can also include a pump 550 and/or a control valve to control the amount and rate at which additive is added to the water from the water supply 520.

The aqueous composition supply 504 can include an aqueous composition control valve 526. The aqueous composition control valve 526 can be configured to control the rate at which aqueous composition is added to the interior volume 510 of the vessel 502. In various embodiments, the aqueous composition control valve 526 can include a mixing element which can mix the aqueous composition to ensure the additive and water are properly mixed. Aqueous composition leaving the aqueous composition supply 504 can pass through the outlet 528 and into the vessel 502.

In some embodiments, an amount of aqueous composition to be added to the interior volume 510 (or an expected amount) can be calculated prior to prior to the start of any moisture addition. For example, the amount of aqueous composition being added can be calculated based on the amount of the feed stuffs being processed. In some embodiments, the amount of aqueous composition can be calculated based on the amount of the feed stuffs along with the moisture content of the feed stuffs prior to prior to moisture addition (e.g., starting moisture content). In some embodiments, the amount of aqueous composition to be added to the interior volume 510 can be calculated based on the amount of the feed stuffs, the starting moisture content, and the desired moisture content of the feed stuffs after conditioning or mixing the feed stuffs with the aqueous composition.

The aqueous composition supply 504 can be configured to send or receive signals from the control circuit 508. In some embodiments, the aqueous composition supply 504 can receive control signals from the control circuit 508 related to delivering the aqueous composition to the vessel. For example, the aqueous composition supply 504 can receive a signal from the control circuit 508 instructing the aqueous composition supply 504 to supply the aqueous composition to the vessel. The signal from the control circuit 508 can include information regarding the amount of aqueous composition to supply, the rate at which it should be supplied, and the timing of when it should be supplied.

### Particulate Material/Feedstuff Material Supply System

The feedstuff supply system 506 can be configured to add a feedstuff into the interior volume 510. The feedstuff supply system 506 can control the rate at which grain is added into the vessel 502 and the amount of feedstuffs that are added into the vessel 502. In some embodiments, the feedstuff supply system 506 can include a delivery mechanism 530, such as a conveyor or a chute which can transport feedstuffs from a location, such as a storage location, to the vessel 502.

In some embodiments, the aqueous composition addition system 500 can include a second moisture sensor 532. The second moisture sensor 532 can be configured to measure the moisture content of feed stuffs prior to entering the vessel 502, such as to calculate the amount of aqueous composition needed to be added to reach a desired moisture content of the feedstuff once it has been mixed with the aqueous composition within the interior volume 510. In various embodiments, the second moisture sensor 532 can include a microwave moisture sensor or a capacitance moisture sensor. The second moisture sensor 532 can be configured to send a signal which represents the moisture content of the feedstuff prior to entering the vessel 502 or prior to being mixed with the aqueous composition. The moisture content of feed stuffs prior to entering the interior volume 510 can be about 4, 5, 6, 7, 8, 9, 10, 11, 12, 13, 14, 15, 16, 17, 18, 19, 20, 21 or 22 percent by weight, or can fall within a range between any of the foregoing. In some embodiments, the feedstuff supply system 506 can be configured to deliver a known amount of the feed stuffs to the interior volume 510, such as a desired weight or volume of grain. The moisture content of feed stuffs after moisture addition with a system herein can be about 12, 13, 14, 15, 16, 17, 18, 19, 20, 21, 22, 24, 26, 28, 30, 32, 34, 36, or 38 percent by weight. In various embodiments, an amount of moisture can be added that is sufficient to raise the moisture content on a percent by weight basis by at least 1, 2, 3, 4, 6, 7, 8, 9, 10, 11, 12, 13, 14, 15, 16, 17, or 18 percentage points (for example, wherein changing the moisture content of a particulate from 14 wt. % to 22 wt. % represents a change of 8 percentage points), or by an amount falling within a range between any of the foregoing.

The feedstuff supply system 506 can be configured to send or receive signals from the control circuit 508. In some embodiments, the feedstuff supply system 506 can receive control signals from the control circuit 508 related to delivering feed stuffs to the vessel. For example, the feedstuff supply system 506 can receive a signal from the control circuit 508 instructing the feedstuff supply system 506 to supply feedstuff to the vessel. The signal from the control circuit 508 can include information regarding the amount of feedstuff to supply, the rate at which it should be supplied, and the timing of when it should be supplied.

### Control Circuit

Control circuits herein can include various elements to execute operations and/or receive inputs or signals from sensors and buttons and then generate output signals such as to control valves, motors, pumps, display screens and the like. Control circuit components can include components such as application specific integrated circuits (ASICs), microprocessors, microcontrollers, programmable logic controllers (PLCs), and the like.

The control circuit 508 can be configured to send and/or receive signals from the various components within the aqueous composition addition system 500, such as the vessel 502, the aqueous composition supply 504, and the feedstuff supply system 506. The control circuit 508 can be configured to control the aqueous composition supply 504 and/or the feedstuff supply system 506, such as by sending control signals to the aqueous composition supply 504 or the feedstuff supply system 506. The control circuit 508 can monitor the rate and amount of aqueous composition being added to the vessel 502. The control circuit 508 can control the aqueous composition supply 504 to control the amount of aqueous composition added to the vessel 502 and the rate at which aqueous composition is added to the vessel 502, such as by controlling the aqueous composition control valve 526. Similarly, the control circuit 508 can also monitor the rate and amount of the feedstuffs being added to the vessel 502. The control circuit 508 can control the feedstuff supply system 506 to control the amount of the feed stuffs added to the vessel 502 and the rate at which the feedstuffs are added to the vessel 502.

In some embodiments, the control circuit 508 can also include a GPS or other geolocation chip or circuit. The control circuit 508 can log its position at various time points, such as at the start or conclusion of various processing steps described herein. In various embodiments, the control circuit 508 can be configured to receive a signal from the first moisture sensor 512 regarding the moisture content of the material within the vessel 502. The control circuit 508 can change or adjust the addition of aqueous composition to the vessel 502 based on the received signal from the first moisture sensor 512. In some embodiments, the control circuit 508 can be configured to control the aqueous composition supply 504 to increase or decrease the rate at which aqueous composition is being added to the vessel 502, or increase or decrease the total amount of aqueous composition being added to the vessel 502.

The control circuit 508 can compare the actual moisture content of the material in the interior volume 510 at a specific time point to an expected moisture range or a predicted moisture range at the same time point. The predicted moisture range can be an estimated or calculated range of moisture that is expected and/or acceptable for the material to be within given the time point in the overall moisture addition or conditioning process. The predicted range can vary over time considering the contents that are being added to the vessel. The control circuit 508 can determine when the moisture content of the material is not within the predicted range. The predicted moisture range can be based off of the systems previous batches of conditioning the feed stuffs, such that the control circuit 508 can track the moisture content of the material as aqueous composition and/or feed stuffs are added to a vessel and predict how future batches of material are likely to react.

The predicted moisture range can include a high moisture limit and a low moisture limit. The intended moisture of the material inside the vessel 502 can be intended to be below the high moisture limit and above the low moisture limit. The high moisture limit and the low moisture limit can increase over time as aqueous composition is added to the vessel 502.

In some embodiments, the magnitude of the gap between the high moisture limit and the low moisture limit can decrease as time passes and as the process nears the end. This can promote accurately finishing at the precise desired moisture content. In some embodiments, the magnitude of the gap between the high moisture limit and the low moisture limit can contract by at least 5%, 10%, 20%, 30%, 40%, 50%, 80%, or 90% over the time span of the moisture addition process. In some embodiments, the high moisture limit can increase over time until it hits the target moisture amount and then can remain at the target moisture amount until the end of the moisture addition process (in that way the high moisture limit may never rise above the target moisture amount).

However, in some embodiments, the magnitude of the gap between the high moisture limit and the low moisture limit can remain substantially constant throughout the moisture addition process.

In various embodiments, the rate or amount of aqueous composition or feed stuffs being added to the vessel 502 can be changed. Changes to the rate or amount of aqueous composition or grain being added to the vessel 502 can affect the predicted moisture range, such as changes as a result of the measured moisture content being outside of the predicted moisture range. For example, if the rate at which aqueous composition is being added is decreased, the predicted moisture content of the materials within vessel 502 can be decreased to account for the decreased rate of aqueous composition being added.

In some embodiments, the control circuit 508 can include a user interface 534. The user interface 534 can display information to a user, such as the predicted moisture content and/or a predicted average particle size.

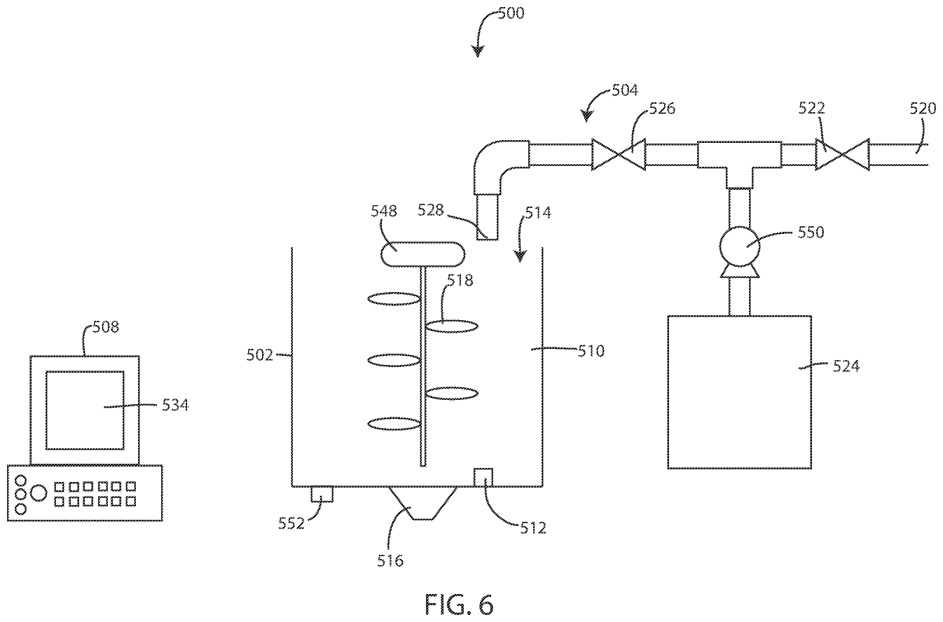

FIG. 6 shows a schematic view of an aqueous composition addition system 500. The aqueous composition addition system 500 shown in FIG. 6 includes a vessel, an aqueous composition supply, and a control circuit. Similar to the embodiment shown in FIG. 5, the control circuit 508 can be configured to monitor the moisture content of material in the vessel 502 with a first moisture sensor 512.

In some embodiments, such as the embodiment shown in FIG. 6, the entire amount of feed stuffs can be deposited into the interior volume of the vessel 502 prior to adding any aqueous composition to the vessel 502. In some embodiments, the feedstuffs can be mixed and then the moisture can be measured with moisture sensor 512 prior to any aqueous composition being added to the vessel 502. The outlet 516 can be closed to ensure the feed stuffs do not exit the vessel 502 while the aqueous composition is being added and the material is being mixed.

The control circuit 508 can be configured to adjust or change the aqueous composition supply 504 in response to the moisture content within the vessel 502 varying from the predicted moisture range. For example, if the moisture content within the vessel 502 is below the predicted moisture range, the control circuit 508 can increase the aqueous composition input from the aqueous composition supply 504. In contrast, if the moisture content within the vessel is above the predicted moisture range, the control circuit 508 can decrease or terminate aqueous composition input from the aqueous composition supply 504 until the measured moisture content returns to the predicted moisture range.

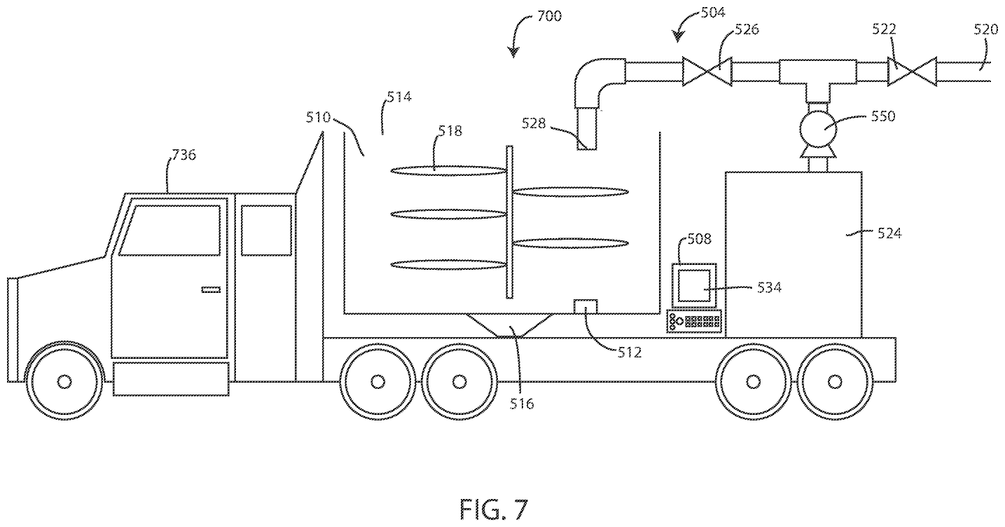

FIG. 7 shows a schematic view of a mobile moisture addition system 700 (or mobile unit), according to an embodiment. In some embodiments, the moisture addition system 700 can include a motor vehicle 736. The mobile moisture addition system 700 can include some or all of the components described with respect to FIGS. 5 and 6, but on a mobile platform. Similar to the embodiment shown in FIG. 6, the mobile moisture addition system 700 can include a vessel 502, an aqueous composition supply 504, and a control circuit 508. The control circuit 508 can control the aqueous composition supply 504 in response to the moisture content within the vessel 502 varying from the predicted range. The vessel 502, the aqueous composition supply 504, and the control circuit 508 can be mounted on the motor vehicle 736, such that the moisture addition system 700 can be easily transported from one location to another. In various embodiments, the vessel 502 can remain mounted on the motor vehicle 736 while feed stuffs within the vessel 502 are being conditioned. In some embodiments, the mobile moisture addition system 700 can also include a scale in order to capture the weight of the vessel 502 and the contents therein. In some embodiments, some components shown in FIG. 7 may be off the vehicle 736 and in wireless communication. For example, the control circuit 508 and/or other components may be off the vehicle 736. Various pieces of information can be exchanged wirelessly between components including, for example, a load size.

### Total Mixed Ration

In some cases, animals may be feed with a total mixed ration (TMR). A total mixed ration is the result of weighing and blending all feed stuffs into a complete ration which provides adequate nourishment for a given animal and may be their sole source of feed. The total mixed ration for an animal can include forage materials, grains, protein supplements, and the like. Components such as forage materials, grains, etc. can be in the form of relatively large particulates along with some portion that may be in the form of fine particulates. The total mixed ration can also include components that may be in the form of fine particulates such as feed supplements, minerals, vitamins, and the like. When all the ingredients are mixed together, an animal is less able to select individual ingredients (sorting) and therefore more likely to get a balanced ration with each bite. The use of a TMR can also promote the ability to feed a variety of different feed stuffs that animals may not eat if fed individually.

TMRs can be formulated based on the calculated total nutritional needs of the animal in combination with the types of feed stuffs that are available in a given area at a given point in time. Control of moisture content of a TMR is extremely important because of its impact on the total energy provided by a given weight of food material. All other things being equal, a TMR having a moisture content of 40% by weight has more energy and nutrients per pound than a TMR having a moisture content of 60% by weight. If animals are always fed a given amount of a TMR as measured by weight, since moisture content provides no calories or nutrients, it can be seen that feeding an animal a TMR that has a moisture content too high will result in under-nourishment of the animal whereas feed an animal a TMR that has a moisture content too low will result in over-nourishment of the animal. Both under-nourishment and over-nourishment are extremely bad for the health and productivity of animals.

In practice, after a TMR is formulated for a group of animals, the ingredients can be weighed and then mixed together. Mixing time can vary based on factors such as the mixer-type, the total amount of feed in the mixer, mixer condition, etc. The mixed TMR can be dispensed in a feed bunk (such as a fence-line bunk) daily, twice daily, or at a greater frequency. In some embodiments, mixing can take place in a mobile unit (such as a feed wagon or mix truck) that can perform mixing while moving between the area where it receives its load and the location of the feed bunks (or other structure) into which the TMR is placed for the animals to feed on.

As a part of the TMR preparation process, moisture content of the TMR can be adjusted. In some cases, TMR formulations that are too dry can result in an undesirable degree of sorting by the animals. In various embodiments, the TMR can be between 20 and 80 percent moisture, or between 30 and 60 percent moisture, or between 40 and 60 percent moisture, or between 45 and 55 percent moisture, or between 30 and 40 percent moisture. In various embodiments, the TMR can have a moisture content of about 20, 25, 30, 35, 40, 45, 50, 55, 60, 65, 70, 75 or 80 percent moisture or an amount falling within a range between any of the foregoing.

TMR can have a particle size distribution that is optimal for the animals. A piece of equipment known as the Penn State Particle Size Separator (PSPSS), which is essentially a three-sieve system with apertures of 19 mm, 8 mm, and 1.18 mm, can be used to assess the particle size distribution of a TMR. In some embodiments, using a PSPSS, the TMR can include 3 to 8 percent of feed on the top screen (X>19 mm), 30 to 50 percent of feed on the middle screen (19 mm>X>8 mm), 30 to 50 percent of feed on the lower screen (8 mm>X>1.18 mm), and less than 20 percent of feed (the smallest particles) on the bottom pan (X<1.18 mm).

Systems and apparatus herein can be used in the preparation of a TMR. In particular, systems and apparatus herein can be used to accurately measure moisture content in a TMR, and adjust the same, despite variations in the moisture content of feedstuff inputs and despite the presence of free moisture allowing for extremely consistent TMR batches and therefore extremely consistent energy and nutrition uptake by animals for enhance productivity and health of such animals.

In various embodiments, certain components that may be at least partly in the form of fine particulates can be added to the TMR to ensure optimal nutrition and health for the animals. The fine particulates can include feed supplements, minerals, vitamins, and/or active agents such as pharmacological agents. These can be costly ingredients added to the animal's feedstuff that can get left behind by animals exhibiting feed sorting behavior. Some of the other feed components (grain, forage, etc.) may also be partly in the form of fine particulates. To prevent feed sorting behavior, it is desirable for the fine particulates to form an agglomeration with the coarser particulates that are part of the TMR. This also serves to ensure the proper amount of the fine particulates is being fed to each animal.

Some of the fine particulates herein can include hydrophobic particles (such as particles with a surface energy as measured by contact angle theta of 90, 100, 110, 120, 130, 140, 150, 160, 170, 175, 178, 179, or 180 degrees, or a contact angle falling within a range wherein the upper and lower bounds of the range can be any of the preceding such as 90 to 180 degrees), ionophores to increase feed efficiency by decreasing the risk of acidosis and bloat, vitamins to correct deficiencies in the feed, prebiotics to remove harmful pathogens from the animal's digestive system, probiotics to control the intestinal microflora, and/or enzymes to improve digestion. For example, the fine particulates can include anionic salts, Aspergillus oryzae, biotin, calcium propionate, protected choline, magnesium oxide, methionine hydroxy analog, monensin (RUMENSIN®, MONOVEDD), melengestrol (MGA), tylosin (TYLAN®), niacin, propylene glycol, sodium bentonite, sodium bicarbonate/sodium sesquicarbonate, yeast culture, yeast, zinc methionine, and silage bacterial inoculants. While not intending to be bound by theory, achieving even distribution of hydrophobic particles can be particularly difficult in a TMR because of the typical addition of water and significant presence of hydrophilic components.

TMR formulations herein can specifically include those with significant fat content. For example, TMR formulations herein can include TMR formulations with at least 1, 1.5, 2, 2.5, 3, 3.5, 4, 5, 6, 7, 8, 9, 10, or 15 wt. % (on a dry matter basis) fat content or more, or an amount falling within a range between any of the foregoing.

Systems with Batch Boxes

In various embodiments, systems herein can also include the use of other components including, but not limited to, batch boxes and/or standalone mixers. A batch box is a container into which ingredients for a TMR batch can be loaded. The volume of the batch box can be sufficiently large for all components of a truckload of TMR. The contents of the batch box can be dumped into a feed truck, mixer-wagon, or other apparatus with a mixing capability all at once.

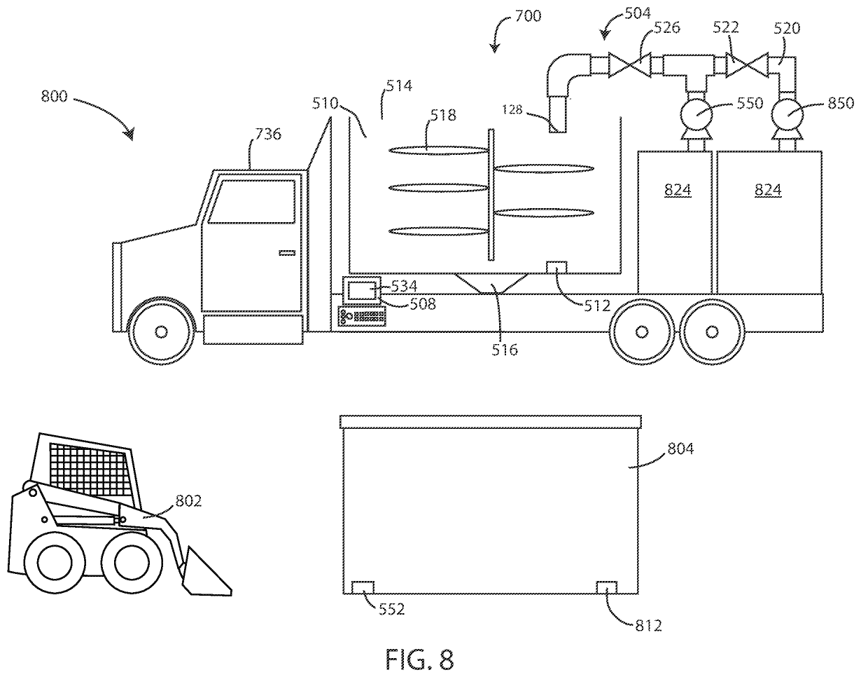

Referring now to FIG. 8, a schematic view is shown of a system 800 including a batch box 804 in accordance with various embodiments herein. The system 800 can include a mobile moisture addition system 700 (or mobile unit). The mobile unit can include elements as described previously herein. In some embodiments, the mobile unit can also include a water supply tank 824 and water pump 850.

In some embodiments, components such as components of a TMR in sufficient quantities for a particular batch can be loaded into a batch box 804. By way of example, a loader 802 (skid steer, front end loader, or the like) can be used to take ingredients in specific amounts and then be deposited into the batch box 804. After all ingredients are deposited into the batch box 804, they can then be put into a device with mixing capability, such as the mobile moisture addition system 700. In various embodiments, the feedstuffs forming a TMR are initially placed into the batch box 804 and then transferred into the mobile moisture addition system 700. Operations of mixing and moisture addition can then take place within the mobile moisture addition system 700, such as while the mobile moisture addition system 700 is moving. In some embodiments, the batch box 804 can include a moisture sensor 812 therein (such as any of the types of moisture sensors described herein). However, in some embodiments, a moisture sensor may be omitted from the batch box 804.

In some embodiments, other sensors can be included in the batch box 804 other than moisture sensors. For example, a weight sensor 552 such as a load cell can be included and can be used by the system, for example, to verify the addition of materials to the batch box 804 and/or to aid in calculations of how much aqueous composition needs to be added to raise moisture content to a target level. In some embodiments, the weight sensor 552 can be used in combination with a moisture sensor 812. For example, the feedstuffs can be added to the batch box and the total amount weighed, then the moisture sensor can take a measurement of the moisture content, which can then be compared to a target moisture content. Then a precise amount of aqueous composition needed for the batch can be calculated in order to raise the moisture content to the target moisture content.

Mobile Scenarios with TMR Mixing

As referenced above, in some embodiments, mixing can take place in a mobile unit (such as a mobile moisture addition system, feed wagon, feed truck, mix truck or other mobile device with mixing capabilities) that can perform mixing while moving between the area where it receives its load of feed and the location of the feed bunks (or other structure) into which the TMR is placed for the animals to feed on.

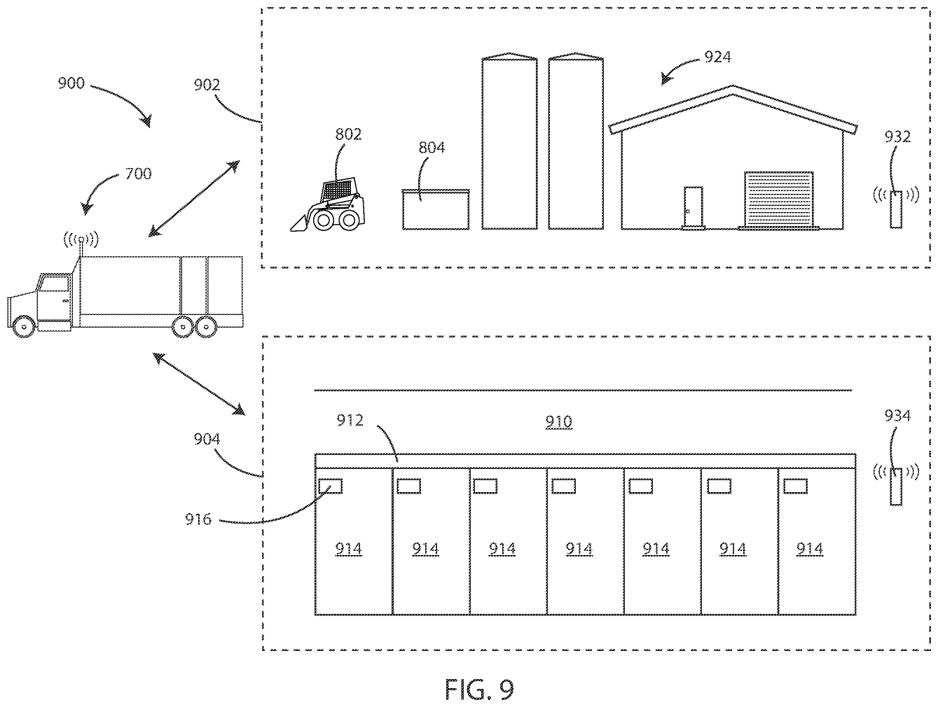

Referring now to FIG. 9 is a schematic diagram of a moisture addition environment 900 in accordance with various embodiments herein. In a first area 902 (or feed supply area or base area), feed materials can be stored at least temporarily in feed storage structures 924. Periodically, the mobile unit 700 can travel to the first area 902 and receive a batch of feed stuffs that will make up a TMR. In some embodiments, the presence of the mobile unit 700 at the first area 902 or base area can be detected and logged using a first proximity sensor 932 and/or using a GPS or other geolocation sensor associated with the mobile unit 700 itself. In various embodiments, a first moisture measurement can be made while the mobile unit 700 is at the first area 902. In various embodiments, moisture measurements can be made while the mobile unit 700 is at the first area 902 and after it has received a load of feedstuff materials and/or after it has received a load of feedstuff materials and an initial amount of a moisture content altering aqueous composition (such as water and an additive described herein).

The mobile unit 700 can then move to an animal containment area 904. In some embodiments, mixing can be performed while the mobile unit 700 is moving between the first area 902 and the animal containment area 904. The animal containment area 904 can include a plurality of pens 914, in some cases with water troughs 916 therein. The animal containment area 904 can further include a feed bunk(s) 912 into which the mobile moisture addition system 700 can deposit TMR allotments. The animal containment area 904 can further include a feed alley 910 through with the mobile moisture addition system 700 can pass when dropping off TMR allotments into the feed bunk(s). In some embodiments, the presence of the mobile unit 700 at the animal containment area 904 can be detected and logged using a second proximity sensor 934 and/or using a GPS or other geolocation sensor associated with the mobile unit 700 itself.

In some embodiments, measurements of moisture can be performed after mixing has been performed and before the feed material is deposited into the feed bunk(s). Various steps can be taken to account for the moisture of the feed material (such as a TMR) that is not as targeted. In some embodiments, the moisture content can be further adjusted in the field (such as additional dry material or additional aqueous composition can be used to “top off” the batch and get the total moisture content closer to the targeted moisture content). In some embodiments, the amount of feed deposited into the feed bunks can be adjusted. In some embodiments, data regarding the variation can be stored and then used when calculating the amount of aqueous composition to add to subsequent batches.

In some embodiments, the amount of feed material deposited into the feed bunks(s) is adjusted based on the final moisture content and how it compares with a predetermined target moisture content. For example, in some embodiments, if the final moisture content is higher than the targeted amount, then the amount of feed material deposited into the feed bunk(s) can be adjusted upwardly to account for the lower energy density of the higher moisture feed material. In some embodiments, if the final moisture content is lower than the targeted amount, then the amount of feed material deposited into the feed bunks(s) can be adjusted downward to account for the higher energy density of the lower moisture feed material.

In some embodiments, data regarding the variation can be stored and then used when calculating the amount of aqueous composition to add to subsequent batches. For example, if it is determined that a 5000-pound batch of a TMR is 2 weight percentage points of moisture below a targeted moisture content, then an additional amount of aqueous composition equal to the 2 weight percentage points of moisture can be added to the next batch in order to account for this discrepancy. Thus, information from previous batches can be used to make final moisture content of future batches more accurate.

### EXAMPLES

### Example 1: Particulate Size Assessment of Various TMR Formulations

In this example, the mass percent of varying particulate sizes of TMR formulations were determined.

In each case, initial moisture content readings of the TMR were obtained. Subsequently, 300 g of the TMR was measured and placed in a zip lock bag and a known amount of moisture was added by percent addition of an aqueous composition. Moisture additions (MA) of 0%, 2%, 6%, and 10% were analyzed. For example, if a 10% moisture addition was desired, 30 g of an aqueous composition was added to the 300 g of the TMR. The TMR with the apportioned moisture addition was then gently shaken 5 times over approximately thirty minutes. After the TMR equilibrated, the final moisture content was determined.

Thereafter, particulate sorting trays T1-T8 were stacked with T1 being on the top and T8 being on the bottom. The T1 tray is designed to hold the largest, coarsest TMR particulates and the T8 tray the smallest, finest TMR particulates. Particulate size values for the trays are as shown below

| Tray | Size Range (Microns) |
|---|---|
| T1 | >=2360 |
| T2 | 1700 to 2360 |
| T3 | 1400 to 1700 |
| T4 | 850 to 1400 |
| T5 | 500 to 850 |
| T6 | 212 to 500 |
| T7 | 150 to 212 |
| T8 | <=150 |

The TMR was placed into the T1 tray and the stacked T1-T8 trays were placed on a RO-TAP® Sieve Shaker 2 minutes with the tap bar running. The stacked trays were then removed, and the mass contained in each tray was measured. The procedural steps above were then repeated 3 times for each TMR having the same moisture addition and the average mass percent for each tray was calculated.

### Mass Percent Averages

TABLE 1

| | TMR | TMR | TMR | TMR | TMR |
|---|---|---|---|---|---|
| | (0% MA) | (2% MA) | (4% MA) | (6% MA) | (10% MA) |
| T1 | 184.40 | 180.35 | 187.95 | 190.00 | 193.00 |
| T2 | 18.95 | 20.55 | 20.70 | 22.30 | 24.90 |
| T3 | 8.90 | 10.00 | 10.20 | 10.70 | 12.70 |
| T4 | 29.0 | 32.75 | 32.75 | 33.75 | 36.70 |
| T5 | 26.45 | 30.70 | 30.35 | 31.20 | 33.50 |
| T6 | 20.05 | 24.55 | 23.75 | 24.00 | 24.10 |
| T7 | 4.65 | 2.35 | 1.75 | 1.40 | 0.80 |
| T8 | 1.50 | 0.80 | 0.30 | 0.20 | 0.10 |

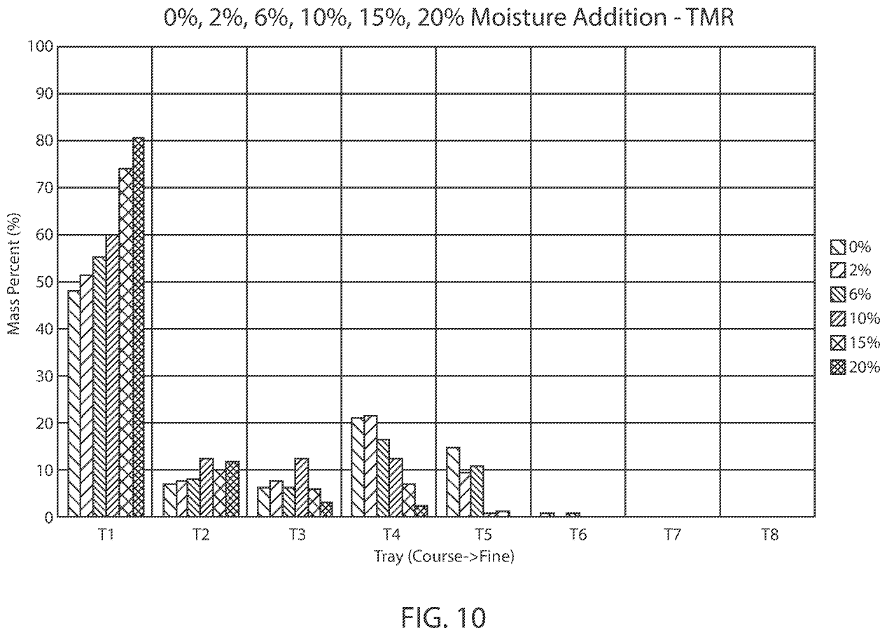

The data for mass percent average are shown in Table 1 above and in FIG. 10.

### Summed Mass Percent Averages

TABLE 2

| | TMR | TMR | TMR | TMR | TMR | TMR |
|---|---|---|---|---|---|---|
| | (0% | (2% | (6% | (10% | (15% | (20% |
| | MA) | MA) | MA) | MA) | MA) | MA) |
| T1- | 62.38 | 68.11 | 77.46 | 86.25 | 88.62 | 96.84 |
| T3 | | | | | | |
| T4- | 37.62 | 31.89 | 22.54 | 13.75 | 8.94 | 3.16 |
| T8 | | | | | | |

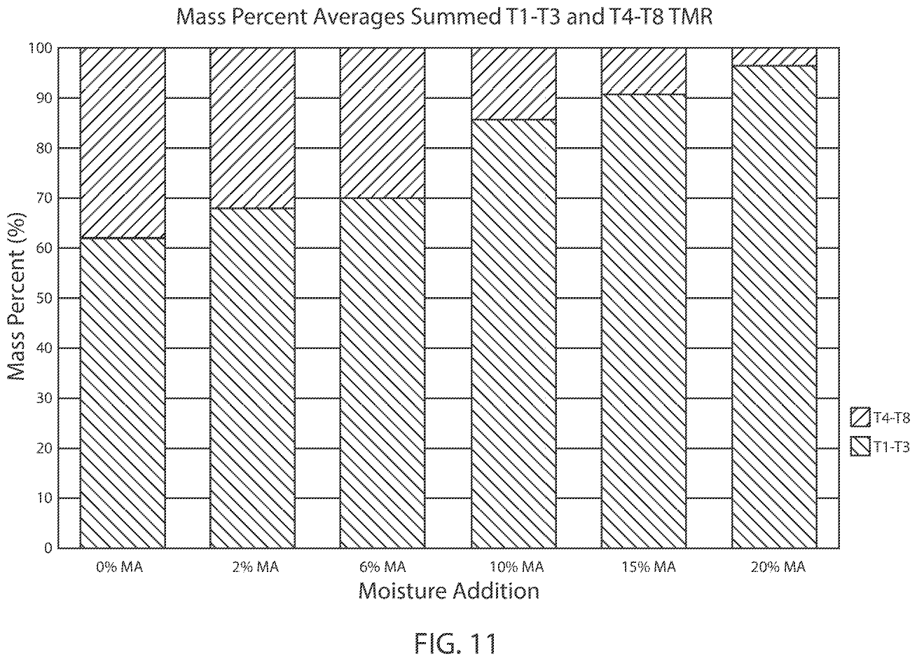

The data for summed mass percent averages are shown in Table 2 above and in FIG. 11.

As FIGS. 10 and 11 illustrate, as the percent of moisture added to the TMR increases, the mass percent of larger particulates increases. This is caused by the aqueous composition added acting to bind the smaller, finer particulates in the TMR to the larger particulates in the TMR thereby creating agglomerations of particulates. The addition at certain concentrations of a yucca-based feed additive containing saponins acting as a surfactant was found to further reduce fines in comparison to just the addition of water.

### Example 2: Distribution of Monensin in TMR

A TMR mixture with monensin and either 4% water plus 2 ounces per ton of a yucca-based feed additive containing saponins (SARSTART LSC GOLD) (Test) or nothing (Control) was placed into a T1 tray and the stacked T1-T8 trays were placed on a RO-TAP® Sieve Shaker 2 minutes with the tap bar running. The stacked trays were then removed. Total mass within trays T1-T5 (larger particles) versus trays T6-T7 was as shown below in Table 3:

TABLE 3

| | | Test + 4% Water + 2 |
|---|---|---|
| | Control | oz/ton Yucca additive |
| | % of Total Weight | % of Total Weight |
| Large Particles | 91.06% | 97.39% |
| (T1-T5 Trays) | | |
| Fines | 8.9% | 2.6% |
| (T6-T7 Trays) | | |

A UV/Vis spectrophotometer quantification technique was used to quantify monensin (poorly soluble in water—monensin sodium solubility is 4.8 mg/L at pH 7) amounts within the different trays. In specific, a representative 1-3-gram TMR sample from each sieve tray, T1-T7, was used to quantify monensin levels in each of the T1-T7 trays. Monensin was extracted from the TMR sample with an equal amount of hexane by weight. The monensin-hexane extract was complexed with an equal amount of ethanol-vanillin (0.0325M) solution by weight and heated for 5 min in a water bath at 60° C. A set of six monensin-vanilla standards were prepared for monensin quantification via a standard curve. Absorbance was measured for all samples and standards with a UV/Vis spectrophotometer at wavelength 520 nm. A TMR hexane extract was used for background subtraction for the samples. The amount of monensin detected was then normalized to the percent mass of each tray.

The data are shown below in Table 4:

TABLE 4

| | | Test + 4% Water + 2 |
|---|---|---|
| | Control | oz/ton Yucca additive |
| | % Monensin | % Monensin |
| Large Particles | 68.7% | 92.1% |
| (T1-T5 Trays) | | |
| Fines | 31.3% | 7.9% |
| (T6-T7 Trays) | | |

The data show that methods and compositions herein such as yucca-based feed additive containing saponins with surfactant properties were highly effective to reduce the relative amount of monensin amongst the fines portion of the feed composition versus the larger particles portion.

### Example 3: Distribution of Components in TMR Over Consumption Period of Time

Testing was performed to evaluate the composition of a TMR (66% Flaked Corn, 22% Corn Silage, 7% dried Distiller's Grain, 5% liquid nutritional supplement) as cattle consumed the ration over time with and without the addition of water and a yucca-based feed additive containing saponins acting as surfactants.

In specific, for a control TMR, the TMR was mixed as normal immediately prior to being deposited in a feed bunk. For a test TMR, a yucca-based feed additive containing saponins (SARSTART LSC GOLD) was added at 2 ounces per ton along with water in an amount of 2 percent by weight and then the TMR was mixed as normal immediately prior to being deposited in a feed bunk. Cattle were allowed to feed from the feed bunk over the course of a day. Samples were collected from the feed bunk at various time points throughout the day as feed was consumed (e.g., less TMR remained in the bunk at later measured time points due to cattle feeding from the bunk). When samples were collected, a cylinder scoop was used to capture the entire volume of the TMR in the bunk from the top to the bottom of the bunk.

The samples were analyzed for various components as shown in TABLES 5 and 6 below:

TABLE 5

| TMR Parameter | CONTROL | CONTROL | CONTROL | CONTROL | | |
|---|---|---|---|---|---|---|
| Measured | 12:35 PM | 2:10 PM | 4:30 PM | Standard Deviation | Average | Rel SD |
| Moisture-* | 27.72 | 26.62 | 25.63 | 1.05 | 26.66 | 3.9% |
| Dry Matter-* | 72.28 | 73.38 | 74.37 | 1.05 | 73.34 | 1.4% |
| Protein, Crude-** | 13.03 | 13.66 | 16.58 | 1.89 | 14.42 | 13.1% |
| ADF-Acid | 7.04 | 7.04 | 6.71 | 0.19 | 6.93 | 2.7% |
| Detergent Fiber-** | | | | | | |
| NEG: Net Energy- | 0.55 | 0.57 | 0.53 | 0.02 | 0.55 | 3.6% |
| Gain-*** | | | | | | |
| NEM: Net Energy- | 0.88 | 0.90 | 0.86 | 0.02 | 0.88 | 2.3% |
| Maintenance-*** | | | | | | |
| TDN: Total | 76.50 | 78.43 | 75.27 | 1.59 | 76.73 | 2.1% |
| Digestible | | | | | | |
| Nutrients-** | | | | | | |
| Fat (EE)-** | 2.07 | 4.03 | 0.60 | 1.72 | 2.23 | 77.1% |
| Calcium-** | 0.63 | 0.64 | 0.60 | 0.02 | 0.62 | 3.3% |
| Phosphorus-** | 0.32 | 0.34 | 0.37 | 0.03 | 0.34 | 7.3% |
| Potassium-* | 0.73 | 0.67 | 0.64 | 0.05 | 0.68 | 6.7% |

* = Units % (As Received)
** = Units % (DM Basis)
*** = Mcal/lb (DM Basis)

TABLE 6

| | | | | TEST | | |
|---|---|---|---|---|---|---|
| TMR Parameter | TEST | TEST | TEST | Standard | | |
| Measured | 11:40 AM | 1:40 PM | 3:40 PM | Deviation | Average | Rel SD |
| Moisture-* | 29.13 | 29.07 | 28.31 | 0.46 | 28.84 | 1.6% |
| Dry Matter-* | 70.87 | 70.93 | 71.69 | 0.46 | 71.16 | 0.6% |
| Protein, Crude-** | 14.06 | 14.00 | 14.31 | 0.16 | 14.12 | 1.2% |
| ADF-Acid | 6.58 | 6.92 | 6.45 | 0.24 | 6.65 | 3.6% |
| Detergent Fiber-** | | | | | | |
| NEG: Net Energy- | 0.58 | 0.58 | 0.58 | 0.00 | 0.58 | 0.0% |
| Gain-*** | | | | | | |
| NEM: Net Energy- | 0.91 | 0.90 | 0.91 | 0.01 | 0.91 | 0.6% |
| Maintenance-*** | | | | | | |
| TDN: Total | 78.97 | 78.53 | 78.81 | 0.22 | 78.77 | 0.3% |
| Digestible | | | | | | |
| Nutrients-** | | | | | | |
| Fat (EE)-** | 4.28 | 4.05 | 4.04 | 0.14 | 4.12 | 3.3% |
| Calcium-** | 0.60 | 0.56 | 0.59 | 0.02 | 0.58 | 3.6% |
| Phosphorus-** | 0.35 | 0.35 | 0.34 | 0.01 | 0.35 | 1.7% |
| Potassium-** | 0.70 | 0.69 | 0.70 | 0.01 | 0.70 | 0.8% |

A comparison of standard deviation values between the control and test TMRs is shown in TABLE 7 below:

TABLE 7

| TMR Parameter Measured | (SD Control/SD Test) × 100 |
|---|---|
| Moisture | 229% |
| Dry Matter | 229% |
| Protein, Crude | 1152% |
| ADF—Acid Detergent Fiber | 79% |
| NEG: Net Energy-Gain | No Variation in Test |
| NEM: Net Energy-Maintenance | 346% |
| TDN: Total Digestible Nutrients | 715% |
| Fat (EE) | 1267% |
| Calcium | 100% |
| Phosphorus | 436% |
| Potassium | 794% |

The data show that standard deviations across the various components analyzed in the TMR were markedly decreased when using the compositions including surfactants herein versus the control TMR. As such, cattle eating the test TMR with the compositions including saponin surfactants ingested a much more consistent TMR regardless of when in the day they were feeding from the bunks. This enhanced consistency is significant as some compounds have a relatively narrow sweet spot for dosing, such as certain fine particulate pharmaceutical compounds.

Further, consistency of fat distribution increased dramatically with the test TMR in comparison with the control TMR. This is particularly important as a number of pharmaceutical compounds may be largely hydrophobic and thus more apt to be associated with fats that with other types of components in the TMR. Taken together, this data show a remarkable ability of methods herein and the compositions used therein to dramatically increase the compositional consistency of TMR fed to cattle under real life conditions.

### Example 4: Effect of Equilibration Time on Fines Reduction

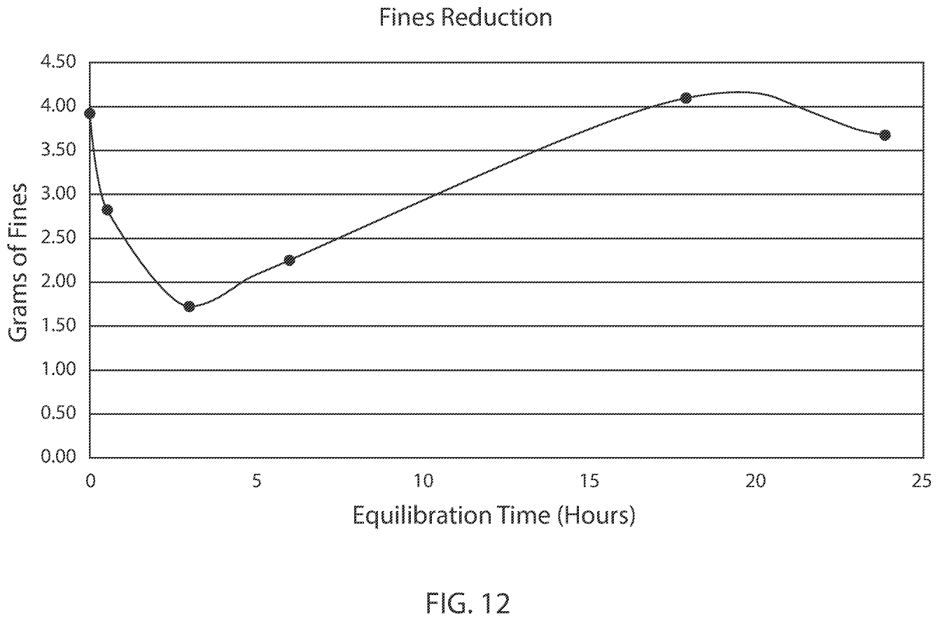

A trial was set up to evaluate the effect of equilibration time on fines reduction. A TMR was mixed together including a fine particulate (monensin), 2 ounces per ton of yucca-based feed additive containing saponins (SARSTART LSC GOLD) and 4% water. After mixing, the composition was allowed to equilibrate for varying amounts of time (from 0 to 24 hours) at room temperature. Then the TMR was placed into the T1 tray and the stacked T1-T8 trays were placed on a RO-TAP® Sieve Shaker for 2 minutes with the tap bar running. The stacked trays were then removed, and the mass contained in each tray was measured. The total amount of mass in the T7 and T8 trays (fines) was determined. The results are shown in FIG. 12. The results show that while there is an initial reduction in fines with some amount of equilibration time, the amount of fines goes back up with longer equilibration times. As the amount of time that passes starting with the addition of the yucca-based feed additive containing saponins to the time the TMR is fed to cattle is significant in achieving optimal fines reduction.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration to. The phrase “configured” can be used interchangeably with other similar phrases such as arranged and configured, constructed and arranged, constructed, manufactured and arranged, and the like.

The technology has been described with reference to various specific and preferred embodiments and techniques. However, it should be understood that many variations and modifications may be made while remaining within the spirit and scope of the technology. As such, the embodiments of the present technology described herein are not intended to be exhaustive or to limit the technology to the precise forms disclosed in the following detailed description. Rather, the embodiments are chosen and described so that others skilled in the art can appreciate and understand the principles and practices of the present technology.

1-26. (canceled)

27. A method of increasing consistency of fine particulate dosing to animals exhibiting feed sorting behavior comprising:
adding total mixed ration components to a vessel;
adding fine particulates to the vessel;
adding an aqueous composition to the vessel;
mixing the aqueous composition, the fine particulates, and the total mixed ration together to form a mixed total mixed ration; and
providing the mixed total mixed ration to an animal.

28. The method of claim 27, the aqueous composition comprising a surfactant.

29. (canceled)

30. The method of claim 27, the aqueous composition comprising sarsasaponins.

31. The method of claim 27, the aqueous composition comprising a yucca extract.

32. The method of claim 27, wherein mixing the aqueous composition, the fine particulates, and the total mixed ration together to form a mixed total mixed ration in the presence of the aqueous composition agglomerates particles to increase the average particle size within the total mixed ration.

33. The method of claim 27, wherein mixing the aqueous composition, the fine particulates, and the total mixed ration together to form a mixed total mixed ration in the presence of the aqueous composition increases the average particle size within the total mixed ration by at least about 10 percent.

34. The method of claim 27, wherein mixing the aqueous composition, the fine particulates, and the total mixed ration together to form a mixed total mixed ration in the presence of the aqueous composition increases the average particle size within the total mixed ration by at least about 50 percent.

35. (canceled)

36. The method of claim 27, the fine particulates comprising at least one selected from the group consisting of a mineral, a vitamin, an ionophore, a nutritional additive, and a pharmacological agent.

37. The method of claim 27, the fine particulates comprising monensin.

38. The method of claim 27, the fine particulates comprising hydrophobic particles.

39. The method of claim 27, wherein adding the aqueous composition to the vessel is performed in an amount equal to at least 2.5% by weight of the total mixed ration components.

40. The method of claim 27, wherein adding the aqueous composition to the vessel is performed in an amount equal to at least 5% by weight of the total mixed ration components.

41. The method of claim 27, wherein adding the aqueous composition to the vessel is performed in an amount equal to at least 7.5% by weight of the total mixed ration components.

42. The method of claim 27, wherein adding the aqueous composition to the vessel is performed in an amount equal to at least 10% by weight of the total mixed ration components.

43. The method of claim 27, wherein providing the mixed total mixed ration to the animal comprises dispensing the mixed total mixed ration into a feed bunk.

44. The method of claim 27, wherein providing the mixed total mixed ration to the animal is performed no more than 360 minutes after the mixing the aqueous composition, the fine particulates, and the total mixed ration together to form a mixed total mixed ration.

45-48. (canceled)

49. The method of claim 27, the animal comprising a ruminant.

50. The method of claim 27, the animal comprising Bos taurus.

51. The method of claim 27, further comprising selecting an animal exhibiting feed sorting behavior.

52. The method of claim 27, the total mixed ration components comprising from 1 to 15 wt. percent (dry matter basis) fat content.

53-73. (canceled)
